(19) United States
(12) Patent Application Publication
(10) Pub. No.: US 2003/0092632 A1
(43) Pub. Date: May 15, 2003

(54) Sulfated CCR5 peptides for HIV-1 infection

(76) Inventors: Tatjana Dragic, Scarsdale, NY (US); William C. Olson, Ossining, NY (US)

Correspondence Address: John P. White, Cooper & Dunham LLP, 1185 Avenue of the Americas, New York, NY, 10036, US

(21) Appl. No.: 10/086,814

(22) Filed: Feb. 28, 2002

Related U.S. Application Data
(60) Provisional application No. 60/272,203, filed on Feb. 28, 2001.

(51) Int. Cl. A61K 38/08; C07K 7/06; C07K 7/08

(52) U.S. Cl. 514/15; 530/328

(57) ABSTRACT

This invention provides a compound comprising the structure: θαYDINYYTSβλ wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined by peptide bonds in consecutive order and have a sequence identical to the sequence set forth in SEQ ID NO: 1 beginning with the I at position 9 and extending therefrom in the amino terminal direction; wherein β represents from 0 to 14 amino acids, with the proviso that if there are more than 2 amino acids, they are joined by peptide bonds in consecutive order and have a sequence identical to the sequence set forth in SEQ ID NO: 1 beginning with the E at position 18 and extending therefrom in the carboxy terminal direction; wherein θ represents an amino group or an acetylated amino group; wherein λ represents a carboxyl group or an amidated carboxyl group; wherein all of α,Y,D,I,N,Y,Y,T,S and β are joined together by peptide bonds; further provided that at least two tyrosines in the compound are sulfated.

The invention disclosed herein was made with Government support under NIH Grant Nos. R01Al43847 (T.D.) and R01DK54718 (T.P.S.) from the Department of Health and Human Services. Accordingly, the government has certain rights in this invention.

Throughout this application, various publications are referenced within parentheses. Disclosures of these publications in their entireties are hereby incorporated by reference into this application to more fully describe the state of the art to which this invention pertains. Full bibliographic citations for these references may be found immediately preceding the claims.

### BACKGROUND OF THE INVENTION

HIV-1 entry into target cells is mediated by the successive interaction of the envelope glycoprotein gp120 with CD4 and a co-receptor belonging to the seven trans-membrane G protein-coupled chemokine receptor family (Berger et al. Ann. Rev. Immunol. 17:657, 1999). Binding of gp120 to CD4 exposes or creates a co-receptor binding site on gp120 (Trkola et al. Nature 384:184, 1996, Wu et al. Nature, 384:179, 1996). CCR5 and CXCR4 are the most physiologically relevant and widely used HIV-1 co-receptors (Zhang and Moore, J. Virol. 73:3443, 1999). CCR5 mediates the entry of R5 isolates and CXCR4 mediates the entry of X4 isolates. R5X4 isolates are able to exploit both co-receptors (Berger et al. Ann. Rev. Immunol. 17:657, 1999). It has been demonstrated that specific amino acids including acidic residues and tyrosines located within the CCR5 amino-terminal domain (Nt, amino acids 2-31) are essential for CCR5-mediated fusion and entry of R5 and R5X4 HIV-1 strains (Dragic et al. J. Virol. 72:279, 1998; Rabut et al. J. Virol. 72:3464, 1998; Farzan et al. J. Virol. 72:1160, 1998; Dorantz et al. J. Virol. 71:6305, 1997). More recently, Farzan et al. demonstrated that tyrosine residues in the CCR5 Nt are sulfated (Farzan et al. Cell 96:667, 1999)

Inhibition of cellular sulfation pathways, including tyrosine sulfation, by sodium chlorate decreased the binding of a gp120/CD4 complex to CCR5⁺ cells (Farzan et al. Cell 96:667, 1999). A number of prior reports had implicated a role for sulfate moieties in HIV-1 entry. Several sulfated compounds, such as dextran sulfate, can inhibit HIV-1 entry by associating with CD4 or gp120 (Baeuerle and Huttner J. Cell Biol 105:2655, 1987; Baba et al. Proc. Natl. Acad. Sci. USA 85:6132, 1998). Sulfated proteoglycans have been shown to bind to HIV-1 gp120 at or near its third variable (V3) loop, which also determines co-receptor usage (Roderiquez et al. J. Virol. 69:2233, 1995; Hwang et al. Science 253:71, 1991). It is therefore conceivable that sulfo-tyrosines in the CCR5 Nt also interact with gp120, increasing its affinity for CCR5. The reduction in gp120/CD4 binding caused by the pre-treatment of target cells with sodium chlorate, however, cannot be formally attributed to a reduction in CCR5 tyrosine sulfation since chlorate can inhibit the sulfation of both tyrosines and proteoglycans.

The region of the CCR5 Nt spanning amino acids 2-18 contains residues that are critically important for viral entry (Dragic et al. J. Virol. 72:279, 1998; Rabut et al. J. Virol. 72:3464, 1998; Farzan et al. J. Virol. 72:1160, 1998; Dorantz et al. J. Virol. 71:6305, 1997). We previously demonstrated that tyrosines at positions 3, 10 and 14 were required for optimal co-receptor function, whereas the Tyr15Phe substitution had little effect on entry (Rabut et al. J. Virol. 72:3464, 1998). Taken together, these findings suggested that HIV-1 entry may be critically dependent upon sulfation of Tyr-3, -10 and -14, but not Tyr-15. We therefore explored the role of sulfo-tyrosines in positions 3, 10 and 14 by synthesizing peptides corresponding to amino acids 2-18 of the CCR5 Nt and carrying different tyrosine modifications. We first tested the ability of the Nt peptides to inhibit binding of gp120/CD4 complexes and anti-CCR5 MAbs to CCR5⁺ cells. The specific association of certain peptides with gp120/sCD4 complexes or with anti-CCR5 MAbs was further confirmed by surface plasmon resonance (BIAcore) analysis. Inhibition of HIV-1 entry by the CCR5 Nt peptides was also tested. Our results suggest that amino acids 2-18 of the CCR5 Nt compose a gp120-binding site that determines the specificity of the interaction between CCR5 and gp120s from R5 and R5X4 isolates. Post-translational sulfation of the tyrosine residues in the CCR5 Nt is required for gp120 binding and may critically modulate the susceptibility of target cells to HIV-1 infection in vivo.

CCR5's normal physiologic activities involve binding and transducing signals mediated by CC-chemokines, including RANTES, MIP-1α and MIP-1β, which direct activation and trafficking of T cells and other inflammatory cells. As such, CCR5 plays an important role in mediating the inflammatory reaction of diseases such as rheumatoid arthritis and multiple sclerosis. The synovial fluid of rheumatoid arthritis patients is highly enriched in CCR5-expressing T cells (Qin et al. J Clin Invest 101:746, 1998), and CCR5 is the predominant CC chemokine receptor expressed on T cells in the rheumatoid synovium (Gomez-Reino et al. Arthritis Rheum 42:989, 1999). Similarly, infiltration by CCR5-expressing cells is characteristic of plaque lesions in patients with multiple schlerosis (Balashov et al. Proc Natl Acad Sci USA 96:6873, 1999). Such observations provide a rationale for the use of agents that block CCR5 for therapy of inflammatory/autoimmune diseases, including but not limited to arthritis, multiple sclerosis, asthma, psoriasis, autoimmune diabetes, transplant rejection, and atherosclerosis.

### SUMMARY OF THE INVENTION

This invention provides a compound comprising the structure:

θαYDINYYTSβλ

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined by peptide bonds in consecutive order and have a sequence identical to the sequence set forth in SEQ ID NO: 1 beginning with the I at position 9 and extending therefrom in the amino terminal direction; wherein β represents from 0 to 14 amino acids, with the proviso that if there are more than 2 amino acids, they are joined by peptide bonds in consecutive order and have a sequence identical to the sequence set forth in SEQ ID NO: 1 beginning with the E at position 18 and extending therefrom in the carboxy terminal direction; wherein θ represents an amino group or an acetylated amino group; wherein λ represents a carboxyl group or an amidated carboxyl group; wherein all of α,Y,D,I,N,Y,Y,T,S and β are joined together by peptide bonds; further provided that at least two tyrosines in the compound are sulfated.

This invention also provides a compound comprising the structure:

θαYDINYYTSβλ

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined by peptide bonds in consecutive order and have a sequence identical to the sequence set forth in SEQ ID NO: 1 beginning with the I at position 9 and extending therefrom in the amino terminal direction; wherein β represents from 0 to 334 amino acids, with the proviso that if there are more than 2 amino acids, they are joined by peptide bonds in consecutive order and have a sequence identical to the sequence set forth in SEQ ID NO: 1 beginning with the E at position 18 and extending therefrom in the carboxy terminal direction;

wherein θ represents an amino group or an acetylated amino group; wherein λ represents a carboxyl group or an amidated carboxyl group; wherein all of α,Y,D,I,N,Y,Y,T,S and β are joined together by peptide bonds; further provided that at least two tyrosines in the compound are sulfated.

This invention provides a composition which comprises a carrier and an amount of one of the compounds described herein effective to inhibit binding of HIV-1 to a CCR5 receptor on the surface of a CD4+ cell.

This invention provides a method of inhibiting human immunodeficiency virus infection of a CD4+ cell which also carries a CCR5 receptor on its surface which comprises contacting the CD4+ cell with an amount of one of the compounds described herein effective to inhibit binding of human immunodeficiency virus to the CCR5 receptor so as to thereby inhibit human immunodeficiency virus infection of the CD4+ cell.

This invention provides a method of preventing CD4+ cells of a subject from becoming infected with human immunodeficiency virus which comprises administering to the subject an amount of one of the compounds described herein effective to inhibit binding of human immunodeficiency virus to CCR5 receptors on the surface of the CD4+ cells so as to thereby prevent the subject's CD4+ cells from becoming infected with human immunodeficiency virus.

This invention provides a method of treating a subject whose CD4+ cells are infected with human immunodeficiency virus which comprises administering to the subject an amount of one of the compounds described herein effective to inhibit binding of human immunodeficiency virus to CCR5 receptors on the surface of the subject's CD4+ cells so as to thereby treat the subject.

This invention provides a method of identifying an agent which inhibits binding of a CCR5 ligand to a CCR5 receptor which comprises:

(a) immobilizing one of the compounds described herein on a solid support;

(b) contacting the immobilized compound from step (a) with sufficient detectable CCR5 ligand to saturate all binding sites for the CCR5 ligand on the immobilized compound under conditions permitting binding of the CCR5 ligand to the immobilized compound so as to form a complex;

(c) removing any unbound CCR5 ligand;

(d) contacting the complex from step (b) with the agent; and

(e) detecting whether any CCR5 ligand is displaced from the complex, wherein displacement of detectable CCR5 ligand from the complex indicates that the agent binds to the compound so as to thereby identify the agent as one which inhibits binding of the CCR5 ligand to the CCR5 receptor.

This invention provides a method of identifying an agent which inhibits binding of a CCR5 ligand to a CCR5 receptor which comprises:

(a) contacting one of the compounds described herein with sufficient detectable CCR5 ligand to saturate all binding sites for the CCR5 ligand on the compound under conditions permitting binding of the CCR5 ligand to the compound so as to form a complex;

(b) removing any unbound CCR5 ligand;

(c) measuring the amount of CCR5 ligand which is bound to the compound in the complex;

(d) contacting the complex from step (a) with the agent so as to displace CCR5 ligand from the complex;

(e) measuring the amount of CCR5 ligand which is bound to the compound in the presence of the agent; and

(f) comparing the amount of CCR5 ligand bound to the compound in step (e) with the amount measured in step (c) wherein a reduced amount measured in step (e) indicates that the agent binds to the compound so as to thereby identify the agent as one which inhibits binding of the CCR5 ligand to the CCR5 receptor.

This invention also provides a method of identifying an agent which inhibits binding of a CCR5 ligand to a CCR5 receptor which comprises:

(a) immobilizing one of the compounds described herein on a solid support;

(b) contacting the immobilized compound from step (a) with the agent and sufficient detectable CCR5 ligand to saturate all binding sites for the CCR5 ligand on the compound under conditions permitting binding of the CCR5 ligand to the immobilized compound so as to form a complex;

(c) removing any unbound CCR5 ligand;

(d) measuring the amount of detectable CCR5 ligand which is bound to the immobilized compound in the complex;

(e) measuring the amount of detectable CCR5 ligand which binds to the immobilized compound in the absence of the agent;

(f) comparing the amount of CCR5 ligand which is bound to the immobilized compound in step (e) with the amount measured in step (d), wherein a reduced amount measured in step (d) indicates that the agent binds to the compound so as to thereby identify the agent as one which inhibits binding of the CCR5 ligand to the CCR5 receptor.

This invention also provides a method of identifying an agent which inhibits binding of a CCR5 ligand to a CCR5 receptor which comprises:

(a) contacting one of the compounds described herein with the agent and sufficient detectable CCR5 ligand to saturate all binding sites for the CCR5 ligand on the compound under conditions permitting binding of the CCR5 ligand to the compound so as to form a complex;

(b) removing any unbound CCR5 ligand;

(c) measuring the amount of detectable CCR5 ligand which is bound to the compound in the complex;

(d) measuring the amount of detectable CCR5 ligand which binds to the compound in the absence of the agent;

(e) comparing the amount of CCR5 ligand which is bound to the compound in step (c) with the amount measured in step (d), wherein a reduced amount measured in step (c) indicates that the agent binds to the compound so as to thereby identify the agent as one which inhibits binding of the CCR5 ligand to the CCR5 receptor.

This invention provides a method of identifying an agent which inhibits binding of a CCR5 ligand to a CCR5 receptor which comprises:

a) immobilizing one of the compounds described herein on a solid support;

b) contacting the immobilized compound from step a) with the agent dissolved or suspended in a known vehicle and measuring the binding signal generated by such contact;

c) contacting the immobilized compound from step a) with the known vehicle in the absence of the compound and measuring the binding signal generated by such contact;

d) comparing the binding signal measured in step b) with the binding signal measured in step c), wherein an increased amount measured in step b) indicates that the agent binds to the compound so as to thereby identify the agent as one which binds to the CCR5 receptor.

This invention provides a method of obtaining a composition which comprises:

(a) identifying a compound which inhibits binding of a CCR5 ligand to a CCR5 receptor according to one of the above methods; and

(b) admixing the compound so identified or a homolog or derivative thereof with a carrier.

This invention provides a compound having the structure:

Δ-(αYDINYYTSβλ)_{π}

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction; wherein β represents from 0 to 14 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction; wherein λ represents a carboxyl group or an amidated carboxyl group; wherein all of α,Y,D,I,N,Y,Y,T,S and β are joined together by peptide bonds, further provided that at least two tyrosines in the compound are sulfated, wherein π is an integer from 1 to 8, Δ is a polymer, and the solid line represents up to 8 linkers which attach the structure in parentheses to Δ.

This invention also provides a compound having the structure:

(θαYDINYYTSβ)_{π}-Δ

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction; wherein β represents from 0 to 14 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction; wherein θ represents an amino group or an acetylated amino group; wherein all of α,Y,D,I,N,Y,Y,T,S and β are joined together by peptide bonds, further provided that at least two tyrosines in the compound are sulfated, wherein π is an integer from 1 to 8, Δ is a polymer, and the solid line represents up to 8 linkers which attach the structure in parentheses to Δ.

This invention provides a compound having the structure:

Δ-(αYDINYYTSβλ)_{π}

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction; wherein β represents from 0 to 334 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction; wherein λ represents a carboxyl group or an amidated carboxyl group; wherein all of α,Y,D,I,N,Y,Y,T,S and β are joined together by peptide bonds,

further provided that at least two tyrosines in the compound are sulfated, wherein π is an integer from 1 to 8, Δ is a polymer, and the solid line represents up to 8 linkers which attach the structure in parentheses to Δ.

This invention also provides a compound having the structure:

(θαYDINYYTSβ)_{π}-Δ

wherein each T represents a threonine, each S represents a serine, each Y represents a tyrosine; each D represents an aspartic acid, each I represents an isoleucine; and each N-represents an asparagine; wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction; wherein β represents from 0 to 334 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction; wherein θ represents an amino group or an acetylated amino group; wherein all of α,Y,D,I,N,Y,Y,T,S and β are joined together by peptide bonds, further provided that at least two tyrosines in the compound are sulfated, wherein π is an integer from 1 to 8, Δ is a polymer, and the solid line represents up to 8 linkers which attach the structure in parentheses to Δ.

### BRIEF DESCRIPTION OF THE FIGURES

FIG. 1 Effect of peptides on gp120_{JR-FL} binding to CCR5. L1.2-CCR5⁺ cells were incubated with the biotinylated gp120_{JR-FL}/CD4-IgG2 complex in the presence of different concentration of peptides (a) S-3/10/14, S-10/14, S-10, S-14 or (b) P-3/10/14, P-10/14, SR-2/12, SR-10/14, TS-10/14. The extent of complex binding in the absence of peptide was defined as 100% (m.f.i. ˜40±5). Binding in the presence of peptide is expressed as a percentage of control. When CCR5-negative cells were used, binding of the gp120_{JR-FL}/CD4-IgG2 complex was negligible (˜10%, m.f.i. ˜2±1). The values shown are from a representative experiment.

FIG. 2 Binding of the gp120/sCD4 complex to sulfated and phosphorylated peptides.

Biotinylated peptides were immobilized on a sensor chip and their ability to associate with gp120/sCD4 was analyzed by BIAcore. RU values as a function of time were measured in the absence of peptide (gray dotted lines), in the presence of phosphorylated peptide (black dotted lines) or in the presence of sulfated peptide (solid black lines). We performed binding analyses with the following proteins: (a) gp120_{JR-FL}/sCD4, (b) gp120_{JR-FL}, (c) sCD4, (d) DV3gp120_{JR-FL}/sCD4, (e) gp120_{DH123}/sCD4, (f) gp120_{DH123}, (g) gp120_{LAI}/sCD4 and (h) gp120_{LAI}.

FIG. 3 Effect of peptides on MAb binding to CCR5.

L1.2-CCR5⁺ cells were incubated with the anti-CCR5 MAbs in the presence of peptides. The extent of MAb binding in the absence of peptide was defined as 100% (m.f.i. ˜50-400, depending on the MAb). Binding in the presence of peptide is expressed as a percentage of control. When CCR5-negative cells were used, binding of MAbs was negligible (m.f.i. ˜2±1). Each data point represents the mean±s.d. of three replicates.

FIG. 4 Binding of MAbs to sulfated and phosphorylated peptides.

Biotinylated peptides were immobilized on a sensor chip and their ability to associate with anti-CCR5 MAbs was analyzed by BIAcore. RU values as a function of time were measured in the absence of peptide (gray dotted lines), in the presence of phosphorylated peptide (black dotted lines) or in the presence of sulfated peptide (solid black lines). We performed binding analyses with (a) PA8, (b) PA10 and (c) 2D7.

FIG. 5 Effect of peptides on viral entry.

HeLa-CD4⁺CCR⁵+ cells were infected with Nlluc⁺env⁻ pseudotyped with different viral envelopes in the presence of peptides. Luciferase activity (r.l.u.) was mesured 48 h post-infection. The extent of entry in the absence of peptide was defined as 100% (r.l.u.. ˜25,000±9,000). Background r.l.u. values were ˜7±2. Each data point represents the mean±s.d. of three replicates.

FIG. 6 CCR5 Nt peptide sequences and labels

The primary sequence of each peptide is indicated in the left column and the corresponding label is indicated in the right column. Sulfated tyrosine residues are designated by black boxes and white boxes designate phosphorylated tyrosine residues.

### DETAILED DESCRIPTION OF THE INVENTION

The plasmids CD4-IgG2-HC-pRcCMV and CD4-kLC-pRcCMV were deposited pursuant to, and in satisfaction of, the requirements of the Budapest Treaty on the International Recognition of the Deposit of Microorganisms (the “Budapest Treaty”) for the Purposes of Patent Procedure with the American Type Culture Collection (ATCC), 12301 Parklawn Drive, Rockville, Md. 20852 under ATCC Accession Nos. 75193 and 75194, respectively.

The plasmids designated PPI4-tPA-gp120_{JR-FL} and PPI4-tPA-gp120_{LAI} were deposited pursuant to, and in satisfaction of, the requirements of the Budapest Treaty on the International Recognition of the Deposit of Microorganisms for the Purposes of Patent Procedure with the American Type Culture Collection (ATCC), 12301 Parklawn Drive, Rockville, Md. 20852 under ATCC Accession Nos. 75431 and 75432, respectively. These plasmids were deposited with ATCC on Mar. 12, 1993. These eukaryotic shuttle vectors contain the cytomegalovirus major immediate-early (CMV MIE) promoter/enhancer linked to the full-length HIV-1 envelope gene whose signal sequence was replaced with that derived from tissue plasminogen activator. In the vector, a stop codon has been placed at the gp120 C-terminus to prevent translation of gp41 sequences, which are present in the vector. The vector also contains an ampicillin resistance gene, an SV40 origin of replication and a DHFR gene whose transcription is driven by the β-globin promoter.

The monoclonal antibodies PA8, PA10, PA12, and PA14 were deposited pursuant to and in satisfaction of, the requirements of the Budapest Treaty on the International Recognition of the Deposit of Microorganisms for the Purposes of Patent Procedure with the American Type Culture Collection (ATCC), 10801 University Boulevard, Manassas, Va. 20110-2209 on Dec. 2, 1998 under the following Accession Nos.: ATCC Accession No. HB-12605 (PA8), ATCC Accession No.HB-12607 (PA10), ATCC Accession No. HB-12609 (PA12), and ATCC Accession No. HB-12610 (PA14).

As used herein, the following standard abbreviations are used throughout the specification to indicate specific amino acids:

| | |
|---|---|
| A = ala = alanine | R = arg = arginine |
| N = asn = asparagine | D = asp = aspartic acid |
| C = cys = cysteine | Q = gln = glutamine |
| E = glu = glutamic acid | G = gly = glycine |
| H = his = histidine | L = ile = isoleucine |
| L = leu = leucine | K = lys = lysine |
| M = met = methionine | F = phe = phenylalanine |
| P = pro = proline | S = ser = serine |
| T = thr = threonine | W = trp = tryptophan |
| Y = tyr = tyrosine | V = val = valine |
| B = asx = asparagine | |
| or aspartic acid | |
| Z = glx = glutamine | |
| or glutamic acid | |

This invention provides a compound comprising the structure:

θαYDINYYTSβλ

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined by peptide bonds in consecutive order and have a sequence identical to the sequence set forth in SEQ ID NO: 1 beginning with the I at position 9 and extending therefrom in the amino terminal direction; wherein β represents from 0 to 14 amino acids, with the proviso that if there are more than 2 amino acids, they are joined by peptide bonds in consecutive order and have a sequence identical to the sequence set forth in SEQ ID NO: 1 beginning with the E at position 18 and extending therefrom in the carboxy terminal direction;

wherein θ represents an amino group or an acetylated amino group; wherein λ represents a carboxyl group or an amidated carboxyl group; wherein all of α,Y,D,I,N,Y,Y,T,S and β are joined together by peptide bonds; further provided that at least two tyrosines in the compound are sulfated.

In one embodiment of the above compound, α represents less than 9 amino acids. In another embodiment of the above compound, α represents less than 8 amino acids. In another embodiment of the above compound, α represents less than 7 amino acids. In another embodiment of the above compound, a represents less than 6 amino acids. In another embodiment of the above compound, α represents less than 5 amino acids. In another embodiment of the above compound, α represents less than 4 amino acids. In another embodiment of the above compound, α represents less than 3 amino acids. In another embodiment of the above compound, α represents less than 2 amino acids. In another embodiment of the above compound, α represents less than 1 amino acid.

In one embodiment of the above compound, β represents less than 17 amino acids. In one embodiment of the above compound, β represents less than 16 amino acids. In one embodiment of the above compound, β represents less than 15 amino acids. In one embodiment of the above compound, β represents less than 14 amino acids. In one embodiment of the above compound, β represents less than 13 amino acids.

In one embodiment of the above compound, β represents less than 12 amino acids. In one embodiment of the above compound, β represents less than 11 amino acids. In one embodiment of the above compound, β represents less than 10 amino acids. In one embodiment of the above compound, β represents less than 9 amino acids. In one embodiment of the above compound, β represents less than 8 amino acids. In one embodiment of the above compound, β represents less than 7 amino acids. In one embodiment of the above compound, β represents less than 6 amino acids. In one embodiment of the above compound, β represents less than 5 amino acids. In one embodiment of the above compound, β represents less than 4 amino acids. In one embodiment of the above compound, β represents less than 3 amino acids. In one embodiment of the above compound, β represents less than 2 amino acids. In one embodiment of the above compound, β represents less than 1 amino acid.

This invention also provides a compound comprising the structure:

θαYDINYYTSβλ

wherein each Y represents a tyrosine; each D represents an aspartic acid, each I represents an isoleucine; and each N represents an asparagine; wherein α represents from 0 to 9 amino acids, with the proviso that if there are more than 2 amino acids, they are joined by peptide bonds in consecutive order and have a sequence identical to the sequence set forth in SEQ ID NO: 1 beginning with the I at position 9 and extending therefrom in the amino terminal direction; wherein β represents from 0 to 334 amino acids, with the proviso that if there are more than 2 amino acids, they are joined by peptide bonds in consecutive order and have a sequence identical to the sequence set forth in SEQ ID NO: 1 beginning with the E at position 18 and extending therefrom in the carboxy terminal direction;

wherein θ represents an amino group or an acetylated amino group; wherein λ represents a carboxyl group or an amidated carboxyl group; wherein all of α,Y,D,I,N,Y,Y,T,S and β are joined together by peptide bonds; further provided that at least two tyrosines in the compound are sulfated.

In one embodiment of the above compound, β represents less than 300 amino acids. In another embodiment of the above compound, β represents less than 250 amino acids. In another embodiment of the above compound, β represents less than 200 amino acids. In another embodiment of the above compound, β represents less than 150 amino acids. In another embodiment of the above compound, β represents less than 100 amino acids. In another embodiment of the above compound, β represents less than 75 amino acids. In another embodiment of the above compound, β represents less than 50 amino acids. In another embodiment of the above compound, β represents less than 40 amino acids. In another embodiment of the above compound, β represents less than 35 amino acids. In another embodiment of the above compound, β represents less than 30 amino acids. In another embodiment of the above compound, β represents less than 25 amino acids. In another embodiment of the above compound, β represents less than 20 amino acids. In another embodiment of the above compound, β represents less than 19 amino acids. In another embodiment of the above compound, β represents less than 18 amino acids. In another embodiment of the above compound, β represents less than 17 amino acids. In another embodiment of the above compound, β represents less than 16 amino acids. In another embodiment of the above compound, β represents less than 15 amino acids. In another embodiment of the above compound, β represents less than 14 amino acids. In another embodiment of the above compound, β represents less than 13 amino acids. In another embodiment of the above compound, β represents less than 12 amino acids. In another embodiment of the above compound, β represents less than 11 amino acids.

In one embodiment of the above compound, α represents less than 9 amino acids. In another embodiment of the above compound, α represents less than 8 amino acids. In another embodiment of the above compound, α represents less than 7 amino acids. In another embodiment of the above compound, α represents less than 6 amino acids. In another embodiment of the above compound, α represents less than 5 amino acids. In another embodiment of the above compound, α represents less than 4 amino acids. In another embodiment of the above compound, α represents less than 3 amino acids. In another embodiment of the above compound, α represents less than 2 amino acids. In another embodiment of the above compound, α represents less than 1 amino acid.

The CCR5 amino acid sequence is the following and is set forth in SEQ ID NO:1:

| | | |
|---|---|---|
| 1 | MDYQVSSPIYDINYYTSEPCQKINVKQIAARLLPPLYSLV | |
| 41 | FIFGFVGNMLVILILINCKRLKSMTDIYLLNLAISDLFFL | |
| 81 | LTVPFWAHYAAAQWDFGNTMCQLLTGLYFIGFFSGIFFII | |
| 121 | LLTIDRYLAVVHAVFALKARTVTFGVVTSVITWVVAVFAS | |
| 161 | LPGIIFTRSQKEGLHYTCSSHFPYSQYQFWKNFQTLKIVI | |
| 201 | LGLVLPLLVMVICYSGILKTLLRCRNEKKRHRAVRLIFTI | |
| 241 | MIVYFLFWAPYNIVLLLNTFQEFFGLNNCSSSNRLDQAMQ | |
| 281 | VTETLGMTHCCINPIIYAFVGEKFRNYLLVFFQKHIAKRF | |
| 321 | CKCCSIFQQEAPERASSVYTRSTGEQEISVGL | 352 |

The CCR5 nucleotide sequence is the following and is set forth in SEQ ID NO:2:

| | |
|---|---|
| 1 | GAATTCCCCC AACAGAGCCA AGCTCTCCAT CTAGTGGACA |

GGGAAGCTAG CAGCAAACCT

| | |
|---|---|
| 61 | TCCCTTCACT ACAAAACTTC ATTGCTTGGC CAAAAAGAGA |

GTTAATTCAA TGTAGACATC

| | |
|---|---|
| 121 | TATGTAGGCA ATTAAAAACC TATTGATGTA TAAAACAGTT |

TGCATTCATG GAGGGCAACT

| | |
|---|---|
| 181 | AAATACATTC TAGGACTTTA TAAAAGATCA CTTTTTATTT |

ATGCACAGGG TGGAACAAGA

| | |
|---|---|
| 241 | TGGATTATCA AGTGTCAAGT CCAATCTATG ACATCAATTA |

TTATACATCG GAGCCCTGCC

| | |
|---|---|
| 301 | AAAAAATCAA TGTGAAGCAA ATCGCAGCCC GCCTCCTGCC |

TCCGCTCTAC TCACTGGTGT

| | |
|---|---|
| 361 | TCATCTTTGG TTTTGTGGGC AACATGCTGG TCATCCTCAT |

CCTGATAAAC TGCAAAAGGC

| | |
|---|---|
| 421 | TGAAGAGCAT GACTGACATC TACCTGCTCA ACCTGGCCAT |

CTCTGACCTG TTTTTCCTTC

| | |
|---|---|
| 481 | TTACTGTCCC CTTCTGGGCT CACTATGCTG CCGCCCAGTG |

GGACTTTGGA AATACAATGT

| | |
|---|---|
| 541 | GTCAACTCTT GACAGGGCTC TATTTTATAG GCTTCTTCTC |

TGGAATCTTC TTCATCATCC

| | |
|---|---|
| 601 | TCCTGACAAT CGATAGGTAC CTGGCTGTCG TCCATGCTGT |

GTTTGCTTTA AAAGCCAGGA

| | |
|---|---|
| 661 | CGGTCACCTT TGGGGTGGTG ACAAGTGTGA TCACTTGGGT |

GGTGGCTGTG TTTGCGTCTC

| | |
|---|---|
| 721 | TCCCAGGAAT CATCTTTACC AGATCTCAAA AAGAAGGTCT |

TCATTACACC TGCAGCTCTC

| | |
|---|---|
| 781 | ATTTTCCATA CAGTCAGTAT CAGTTCTGGA AGAATTTCCA |

GACATTAAAG ATAGTCATCT

| | |
|---|---|
| 841 | TGGGGCTGGT CCTGCCGCTG CTTGTCATGG TCATCTGCTA |

CTCGGGAATC CTAAAAACTC

| | |
|---|---|
| 901 | TGCTTCGGTG TCGAAATGAG AAGAAGAGGC ACAGGGCTGT |

GAGGCTTATC TTCACCATCA

| | |
|---|---|
| 961 | TGATTGTTTA TTTTCTCTTC TGGGCTCCCT ACAACATTGT |

CCTTCTCCTG AACACCTTCC

| | |
|---|---|
| 1021 | AGGAATTCTT TGGCCTGAAT AATTGCAGTA GCTCTAACAG |

GTTGGACCAA GCTATGCAGG

| | |
|---|---|
| 1081 | TGACAGAGAC TCTTGGGATG ACGCACTGCT GCATCAACCC |

CATCATCTAT GCCTTTGTCG

| | |
|---|---|
| 1141 | GGGAGAAGTT CAGAAACTAC CTCTTAGTCT TCTTCCAAAA |

GCACATTGCC AAACGCTTCT

| | |
|---|---|
| 1201 | GCAAATGCTG TTCTATTTTC CAGCAAGAGG CTCCCGAGCG |

AGCAAGCTCA GTTTACACCC

| | |
|---|---|
| 1261 | GATCCACTGG GGAGCAGGAA ATATCTGTGG GCTTGTGACA |

CGGACTCAAG TGGGCTGGTG

| | |
|---|---|
| 1321 | ACCCAGTCAG AGTTGTGCAC ATGGCTTAGT TTTCATACAC |

AGCCTGGGCT GGGGGT

As used herein, “CCR5” is a chemokine receptor which binds members of the CC group of chemokines and whose amino acid sequence comprises that provided in Genbank Accession Number 1705896 and related polymorphic variants. The nucleotide sequence comprises that provided in Genbank Accession Number X91492. In one embodiment, the above compound may correspond to the extracellular portion of CCR5. The first 31 amino acids of CCR5 correspond to the extracellular portion of CCR5. Accordingly, the extracellular portion extends from the methionine at position number 1 to the arginine at position number 31 of SEQ ID NO:1. In another embodiment, the above compound may correspond to the amino-terminal portion of CCR5. As used herein, “N-terminus” or amino-terminus means the sequence of amino acids spanning the initiating methionine and the first transmembrane region.

As used herein, “H₂N” refers to the N-terminus or amino-terminus. As used herein, “COOH” refers to the C-terminus or carboxy-terminus.

Various tyrosines of the compounds described herein may be sulfated. These include but are not limited to the tyrosines at positions 3, 10 and 14 of amino acid sequence set forth in SEQ ID NO:1. Accordingly, in one embodiment, the tyrosines at positions 10 and 14 are sulfated. In another embodiment, the tyrosines at positions 3 and 14 are sulfated. In another embodiment, the tyrosines at positions 3 and 10 are sulfated. In another embodiment, the tyrosines at positions 3, 10 and 14 are sulfated. Other tyrosines in the sequence set forth in SEQ ID NO:1 may also be sulfated.

This invention provides a composition comprising one of the compounds described herein and a detectable marker attached thereto. In one embodiment of the composition, the detectable marker is biotin. In one embodiment of the composition, the detectable marker is attached at the C-terminus of the compound.

The compounds of the subject invention may also be isolated or purified. In one embodiment the compound is labeled with a detectable marker. As used herein, chemical “labels” include radioactive isotopes, fluorescent groups and affinity moieties such as biotin that facilitate detection of the labeled peptide. Other chemical labels are well-known to those skilled in the art. Methods for attaching chemical labels to peptides are well-known to the skilled artisan.

As used herein, “peptide” and “polypeptide” are used to denote two or more amino acids linked by a peptidic bond between the α-carboxyl group of one amino acid and the α-amino group of the next amino acid. Peptides may be produced by solid-phase synthetic methods that are well-known to those skilled in the art. In addition to the above set of twenty amino acids that are used for protein synthesis in vivo, peptides may contain additional amino acids, including but not limited to hydroxyproline, sarcosine, and γ-carboxyglutamate. The peptides may contain modifying groups including but not limited to sulfate and phosphate moieties. Peptides can be comprised of L- or D-amino acids, which are mirror-image forms with differing optical properties. Peptides containing D-amino acids have the advantage of being less susceptible to proteolysis in vivo.

Peptides may by synthesized in monomeric linear form, cyclized form or as oligomers such as branched multiple antigen peptide (MAP) dendrimers (Tam et al. Biopolymers 51:311, 1999). Nonlinear peptides may have increased binding affinity by virtue of their restricted conformations and/or oligomeric nature. Peptides may also be produced using recombinant methods as either isolated peptides or as a portion of a larger fusion protein that contains additional amino acid sequences.

Peptides may be chemically conjugated to proteins by a variety of well-known methods. Such peptide-protein conjugates can be formulated with a suitable adjuvant and administered parenterally for the purposes of generating polyclonal and monoclonal antibodies to the peptides of interest. Alternatively, unconjugated peptides can be formulated with adjuvant and administered to laboratory animals for the purposes of generating antibodies. Methods for generating and isolating such antibodies are well-known to those skilled in the art.

This invention provides derivatives of the above compound. As used herein, a “derivative” peptide is one whose amino acid sequence is nonidentical to the reference peptide but which possesses functionally similar binding properties. Derivative peptides may also contain N-terminal, C-terminal and/or internal insertions, deletions, or substitutions of amino acids, with the proviso that such insertions, deletions and substitutions do not abrogate the binding properties of the peptide. Derivative peptides include peptides modified with chemical labels to facilitate detection. Derivative peptides include branched and cyclized peptides.

As used herein, “sulfopeptides” are peptides that contain sulfate moieties attached to one or more amino acids, such a tyrosine. In “sulfo-tyrosines”, a sulfate group replaces the para-hydroxyl group located on tyrosine side-chain.

As used herein, “phosphopeptides” are peptides that contain phosphate moieties attached to one or more amino acids, such a tyrosine. In “phospho-tyrosines”, a phosphate group replaces the para-hydroxyl group located on tyrosine side-chain.

This invention provides a composition which comprises a carrier and an amount of one of the compounds described herein effective to inhibit binding of HIV-1 to a CCR5 receptor on the surface of a CD4+ cell.

The carriers include but are not limited to an aerosol, intravenous, oral or topical carrier. Accordingly. The invention provides the above composition adapted for aerosol, intravenous, oral or topical application.

This invention provides the above compositions and a pharmaceutically acceptable carrier. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

As used herein, “composition” means a mixture. The compositions include but are not limited to those suitable for oral, rectal, intravaginal, topical, nasal, opthalmic, or parenteral administration to a subject. As used herein, “parenteral” includes but is not limited to subcutaneous, intravenous, intramuscular, or intrasternal injections or infusion techniques.

As used herein, “administering” may be effected or performed using any of the methods known to one skilled in the art. The methods may comprise intravenous, intramuscular or subcutaneous means. As used herein, “effective dose” means an amount in sufficient quantities to either treat the subject or prevent the subject from becoming infected with HIV-1. A person of ordinary skill in the art can perform simple titration experiments to determine what amount is required to treat the subject.

This invention provides a method of inhibiting human immunodeficiency virus infection of a CD4+ cell which also carries a CCR5 receptor on its surface which comprises contacting the CD4+ cell with an amount of one of the compounds described herein effective to inhibit binding of human immunodeficiency virus to the CCR5 receptor so as to thereby inhibit human immunodeficiency virus infection of the CD4+ cell. As used herein, “inhibits” means that the amount is reduced. In a preferred embodiment, inhibits means that the amount is reduced 100%.

In one embodiment of this method, the CD4+ cell is present in a subject and the contacting is effected by administering the compound to the subject.

This invention provides a method of preventing CD4+ cells of a subject from becoming infected with human immunodeficiency virus which comprises administering to the subject an amount of one of the compounds described herein effective to inhibit binding of human immunodeficiency virus to CCR5 receptors on the surface of the CD4+ cells so as to thereby prevent the subject's CD4+ cells from becoming infected with human immunodeficiency virus.

This invention provides a method of treating a subject whose CD4+ cells are infected with human immunodeficiency virus which comprises administering to the subject an amount of one of the compounds described herein effective to inhibit binding of human immunodeficiency virus to CCR5 receptors on the surface of the subject's CD4+ cells so as to thereby treat the subject.

As used herein, human immunodeficiency virus includes but is not limited to HIV-1, which is the human immunodeficiency virus type-1. HIV-1 includes but is not limited to extracellular virus particles and the forms of HIV-1 found in HIV-1 infected cells.

As used herein, “HIV-1 infection” means the introduction of HIV-1 genetic information into a target cell, such as by fusion of the target cell membrane with HIV-1 or an HIV-1 envelope glycoprotein⁺ cell. The target cell may be a bodily cell of a subject. In the preferred embodiment, the target cell is a bodily cell from a human subject.

As used herein, “inhibiting HIV-1 infection” means the reduction of the amount of HIV-1 genetic information introduced into a target cell population as compared to the amount that would be introduced without the composition.

In the above methods, the compound may be administered by various routes including but not limited to aerosol, intravenous, oral or topical route.

In one embodiment of the above methods, the subject is infected with HIV-1 prior to administering the compound to the subject. In one embodiment of the above methods, the subject is not infected with HIV-1 prior to administering the compound to the subject. In one embodiment of the above methods, the subject is not infected with, but has been exposed to, human immunodeficiency virus.

In one embodiment of the above methods, the effective amount of the compound comprises from about 1.0 ng/kg to about 100 mg/kg body weight of the subject. In another embodiment of the above methods, the effective amount of the compound comprises from about 100 ng/kg to about 50 mg/kg body weight of the subject. In another embodiment of the above methods, the effective amount of the compound comprises from about 1 μg/kg to about 10 mg/kg body weight of the subject. In another embodiment of the above methods, the effective amount of the compound comprises from about 100 μg/kg to about 1 mg/kg body weight of the subject.

The dose of the composition of the invention will vary depending on the subject and upon the particular route of administration used. Dosages can range from 0.1 to 100,000 μg/kg. Based upon the composition, the dose can be delivered continuously, such as by continuous pump, or at periodic intervals. For example, on one or more separate occasions. Desired time intervals of multiple doses of a particular composition can be determined without undue experimentation by one skilled in the art.

As used herein, “effective dose” means an amount in sufficient quantities to either treat the subject or prevent the subject from becoming infected with HIV-1. A person of ordinary skill in the art can perform simple titration experiments to determine what amount is required to treat the subject.

In one embodiment of the above method, the subject is a human being. As used herein, “subject” means any animal or artificially modified animal capable of becoming HIV-infected. Artificially modified animals include, but are not limited to, SCID mice with human immune systems. The subjects include but are not limited to mice, rats, dogs, guinea pigs, ferrets, rabbits, and primates. In the preferred embodiment, the subject is a human being.

This invention provides a vaccine which comprises the compound described herein. Vaccines comprising the sulfopeptides and a suitable adjuvant could be administered to a subject for the purposes of generating antibodies or other immune responses that are of therapeutic or prophylactic value. For example, the vaccines could be administered for the purpose of generating in the subject antibodies that bind CCR5 and inhibit its ability to mediate HIV entry and infection, thereby protecting the subject from HIV infection or disease progression. The vaccines may also comprise a suitable adjuvant. The vaccine may also comprises a suitable carrier.

The subject invention has various applications which includes HIV treatment such as treating a subject who has become afflicted with HIV. As used herein, “afflicted with HIV-1” means that the subject has at least one cell which has been infected by HIV-1. As used herein, “treating” means either slowing, stopping or reversing the progression of an HIV-1 disorder. In the preferred embodiment, “treating” means reversing the progression to the point of eliminating the disorder. As used herein, “treating” also means the reduction of the number of viral infections, reduction of the number of infectious viral particles, reduction of the number of virally infected cells, or the amelioration of symptoms associated with HIV-1. Another application of the subject invention is to prevent a subject from contracting HIV. As used herein, “contracting HIV-1” means becoming infected with HIV-1, whose genetic information replicates in and/or incorporates into the host cells. Another application of the subject invention is to treat a subject who has become infected with HIV-1. As used herein, “HIV-1 infection” means the introduction of HIV-1 genetic information into a target cell, such as by fusion of the target cell membrane with HIV-1 or an HIV-1 envelope glycoprotein⁺ cell. The target cell may be a bodily cell of a subject. In the preferred embodiment, the target cell is a bodily cell from a human subject. Another application of the subject invention is to inhibit HIV-1 infection. As used herein, “inhibiting HIV-1 infection” means reducing the amount of HIV-1 genetic information introduced into a target cell population as compared to the amount that would be introduced without said composition.

This invention provides a method of identifying an agent which inhibits binding of a CCR5 ligand to a CCR5 receptor which comprises:

(a) immobilizing one of the compounds described herein on a solid support;

(b) contacting the immobilized compound from step (a) with sufficient detectable CCR5 ligand to saturate all binding sites for the CCR5 ligand on the immobilized compound under conditions permitting binding of the CCR5 ligand to the immobilized compound so as to form a complex;

(c) removing any unbound CCR5 ligand;

(d) contacting the complex from step (b) with the agent; and

(e) detecting whether any CCR5 ligand is displaced from the complex, wherein displacement of detectable CCR5 ligand from the complex indicates that the agent binds to the compound so as to thereby identify the agent as one which inhibits binding of the CCR5 ligand to the CCR5 receptor.

This invention provides a method of identifying an agent which inhibits binding of a CCR5 ligand to a CCR5 receptor which comprises:

(a) contacting one of the compounds described herein with sufficient detectable CCR5 ligand to saturate all binding sites for the CCR5 ligand on the compound under conditions permitting binding of the CCR5 ligand to the compound so as to form a complex;

(b) removing any unbound CCR5 ligand;

(c) measuring the amount of CCR5 ligand which is bound to the compound in the complex;

(d) contacting the complex from step (a) with the agent so as to displace CCR5 ligand from the complex;

(e) measuring the amount of CCR5 ligand which is bound to the compound in the presence of the agent; and

(f) comparing the amount of CCR5 ligand bound to the compound in step (e) with the amount measured in step (c), wherein a reduced amount measured in step (e) indicates that the agent binds to the compound so as to thereby identify the agent as one which inhibits binding of the CCR5 ligand to the CCR5 receptor.

This invention also provides a method of identifying an agent which inhibits binding of a CCR5 ligand to a CCR5 receptor which comprises:

(a) immobilizing one of the compounds described herein on a solid support;

(b) contacting the immobilized compound from step (a) with the agent and detectable CCR5 ligand under conditions permitting binding of the CCR5 ligand to the immobilized compound so as to form a complex;

(c) removing any unbound CCR5 ligand;

(d) measuring the amount of detectable CCR5 ligand which is bound to the immobilized compound in the complex;

(e) measuring the amount of detectable CCR5 ligand which binds to the immobilized compound in the absence of the agent;

(f) comparing the amount of CCR5 ligand which is bound to the immobilized compound in step (e) with the amount measured in step (d), wherein a reduced amount measured in step (d) indicates that the agent binds to the compound so as to thereby identify the agent as one which inhibits binding of the CCR5 ligand to the CCR5 receptor.

In one embodiment of the above method, the amount of the detectable CCR5 ligand in step (a) and step (e) is sufficient to saturate all binding sites for the CCR5 ligand on the compound.

This invention also provides a method of identifying an agent which inhibits binding of a CCR5 ligand to a CCR5 receptor which comprises:

(a) contacting one of the compounds described herein with the agent and detectable CCR5 ligand under conditions permitting binding of the CCR5 ligand to the compound so as to form a complex;

(b) removing any unbound CCR5 ligand;

(c) measuring the amount of detectable CCR5 ligand which is bound to the compound in the complex;

(d) measuring the amount of detectable CCR5 ligand which binds to the compound in the absence of the agent;

(e) comparing the amount of CCR5 ligand which is bound to the compound in step (c) with the amount measured in step (d), wherein a reduced amount measured in step (c) indicates that the agent binds to the compound so as to thereby identify the agent as one which inhibits binding of the CCR5 ligand to the CCR5 receptor.

In one embodiment of the above method, the amount of the detectable CCR5 ligand in step (a) and step (d) is sufficient to saturate all binding sites for the CCR5 ligand on the compound.

In one embodiment of the above method the solid support is a microtiter plate well. In another embodiment, the solid support is a bead. In a further embodiment, the solid support is a surface plasmon resonance sensor chip. The surface plasmon resonance sensor chip can have pre-immobilized streptavidin. In one embodiment, the surface plasmon resonance sensor chip is a BIAcore™ chip.

In one embodiment of the above methods, the detectable CCR5 ligand is labeled with a detectable marker. In another embodiment of the above methods, the CCR5 ligand is detected by contacting it with another compound which is both capable of detecting the CCR5 ligand and is detectable. The detectable markers include those described above.

This invention provides a method of identifying an agent which inhibits binding of a CCR5 ligand to a CCR5 receptor which comprises:

a) immobilizing one of the compounds described herein on a solid support;

b) contacting the immobilized compound from step a) with the agent dissolved or suspended in a known vehicle and measuring the binding signal generated by such contact;

c) contacting the immobilized compound from step a) with the known vehicle in the absence of the compound and measuring the binding signal generated by such contact;

d) comparing the binding signal measured in step b) with the binding signal measured in step c), wherein an increased amount measured in step b) indicates that the agent binds to the compound so as to thereby identify the agent as one which binds to the CCR5 receptor.

In one embodiment of the above method, the solid support is a surface plasmon resonance sensor chip. In another embodiment of the above method, the binding signal is measured by surface plasmon resonance.

This invention provides a method of obtaining a composition which comprises:

(a) identifying a compound which inhibits binding of a CCR5 ligand to a CCR5 receptor according to one of the above methods; and

(b) admixing the compound so identified or a homolog or derivative thereof with a carrier.

The invention provides agents identified in the screen. Such agents may have utility in treating HIV-1 infection or other CCR5-mediated diseases, which include rheumatoid arthritis, asthma, multiple sclerosis, psoriasis, atherosclerosis and other inflammatory diseases.

In one embodiment of the above methods, the CCR5 ligand is a complex comprising an HIV-1 envelope glycoprotein and a CD4-based protein. The HIV-1 envelope glycoproteins include but are not limited to gp120, gp140 or gp160. The CD4-based proteins include but are not limited to soluble CD4 or CD4-IgG2.

As used herein, “CD4” means the mature, native, membrane-bound CD4 protein comprising a cytoplasmic domain, a hydrophobic transmembrane domain, and an extracellular domain that binds to the HIV-1 gp120 envelope glycoprotein. As used herein, “HIV-1 envelope glycoprotein” means the HIV-1 encoded protein which comprises the gp120 surface protein, the gp41 transmembrane protein and oligomers and precursors thereof. As used herein, “CD4-based protein” means any protein comprising at least one sequence of amino acid residues corresponding to that portion of CD4 which is required for CD4 to form a complex with the HIV-1 gp120 envelope glycoprotein. As used herein, “CD4-IgG2” means a heterotetrameric CD4-human IgG2 fusion protein encoded by the expression vectors deposited under ATCC Accession Numbers 75193 and 75194.

In one embodiment of the above methods, the CCR5 ligand is a chemokine. The chemokines include but are not limited to RANTES, MIP-1α or MIP-1β. As used herein, “RANTES”, “MIP-1α”, and “MIP-1β” denote members of the chemokine superfamily of proteins that direct the activation and migration of leukocytes and other cells involved in the inflammation. RANTES, MIP-1α and MIP-1β are known to bind CCR5 and induce signaling. Their peptide sequences have been described (Wells et al. J. Leukocyte Biology, 59:53-60, 1996).

In one embodiment of the above methods, the CCR5 ligand is an antibody. In one embodiment, the antibody is PA8 (ATCC Accession No. HB-12605). In another embodiment, the antibody is PA10 (ATCC Accession No.12607). In another embodiment, the antibody is PA11 (ATCC Accession No. HB-12608). In another embodiment, the antibody is PA12 (ATCC Accession No. HB-12609).

This invention provides a compound having the structure:

Δ-(αYDINYYTSβλ)_{π}

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction; wherein β represents from 0 to 14 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction; wherein λ represents a carboxyl group or an amidated carboxyl group; wherein all of α,Y,D,I,N,Y,Y,T,S and β are joined together by peptide bonds, further provided that at least two tyrosines in the compound are sulfated, wherein π is an integer from 1 to 8, Δ is a polymer, and the solid line represents up to 8 linkers which attach the structure in parentheses to Δ.

This invention also provides a compound having the structure:

(θαYDINYYTSβ)_{π}-Δ

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction; wherein β represents from 0 to 14 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction; wherein θ represents an amino group or an acetylated amino group; wherein all of α,Y,D,I,N,Y,Y,T,S and β are joined together by peptide bonds,

further provided that at least two tyrosines in the compound are sulfated, wherein π is an integer from 1 to 8, Δ is a polymer, and the solid line represents up to 8 linkers which attach the structure in parentheses to Δ.

This invention provides a compound having the structure:

Δ-(αYDINYYTSβλ)_{π}

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction; wherein β represents from 0 to 334 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal diection; wherein λ represents a carboxyl group or an amidated carboxyl group; wherein all of α,Y,D,I,N,Y,Y,T,S and β are joined together by peptide bonds, further provided that at least two tyrosines in the compound are sulfated, wherein π is an integer from 1 to 8, Δ is a polymer, and the solid line represents up to 8 linkers which attach the structure in parentheses to Δ.

This invention also provides a compound having the structure:

(θαYDINYYTSβ)_{π}-Δ

wherein each T represents a threonine, each S represents a serine, each Y represents a tyrosine; each D represents an aspartic acid, each I represents an isoleucine; and each N represents an asparagine; wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction; wherein β represents from 0 to 334 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction; wherein θ represents an amino group or an acetylated amino group; wherein all of α,Y,D,I,N,Y,Y,T,S and β are joined together by peptide bonds, further provided that at least two tyrosines in the compound are sulfated, wherein π is an integer from 1 to 8, Δ is a polymer, and the solid line represents up to 8 linkers which attach the structure in parentheses to Δ.

The polymer of the above compounds includes but is not limited to the following: a linear lysine polymer; a branched lysine polymers; a linear arginine polymer; a branched arginine polymer; and polyethylene glycol (PEG), a linear acetylated lysine polymer, a branched acetylated lysine polymer, a linear chloroacetylated lysine polymer and a branched chloroacetylated lysine polymer.

The above compounds can be produced by various methods known to those skilled in the art, including but not limited to the following. Methods for producing synthetic multimeric peptides such as multiple antigen peptides, synthetic polymeric constructs, and branched lysine oligopeptides are well known to those skilled in the art (Spetzler and Tam, Int. J. Pept. Prot. Res. 45:78, 1995; Yai et al., J. Virol., 69:320, 1995; Okuda et al., J. Mol. Recognit. 6:101, 1993). For example, radially branched peptides can be produced by performing standard solid-phase peptide synthesis methods using branched lysine skeletons on 4-(oxy-methyl)-phenylactamidomethyl or other suitable solid resin. Peptide chains are elongated in parallel in a stepwise fashion using optimized t-butyloxycarbonyl/benzyl chemistry as described (Sabatier et al., Biochemistry 32:2763, 1993). Peptides are liberated from the resin, purified by reversed-phase chromatography over a C18 or other suitable column and characterized by analytical HPLC and mass spectroscopy.

In another approach, monomeric peptides are synthesized, purified, and then covalently coupled to lysine copolymers using N-succinimidyl maleimido carboxylate chemistry. In another approach, the peptides can also be made in the form of affinity type multimers. For example, peptides may be synthesized with an affinity tag such as biotin. These affinity tagged peptides can then be mixed with affinity ligands capable of binding multimerically, such as streptravidin. Other site-specific ligation chemistries are known to the skilled artisan.

This invention will be better understood from the Experimental Details that follow. However, one skilled in the art will readily appreciate that the specific methods and results discussed are merely illustrative of the invention as described more fully in the claims that follow thereafter.

### EXPERIMENTAL DETAILS

A. Materials

Purified recombinant CD4-IgG2 protein was produced by Progenics Pharmaceuticals, Inc. from plasmids CD4-IgG2-HC-pRcCMV and CD4-kLC-pRcCMV as described (Allaway et al. AIDS Res. Hum. Retroviruses 11:533, 1995). Soluble CD4 is commercially available (NEN Life Science Products, Boston, Mass.). Anti-CCR5 MAb 2D7 was purchased from Pharmingen (San-Diego, Calif.).

The plasmids designated PPI4-tPA-gp120_{JR-FL}-V3⁽⁻⁾ and PPI4-tPA-gp120_{DH123} were prepared as described (Hasel et al, U.S. Pat. Nos. 5,869,624 and 5,886,163). Monomeric gp120 glycoproteins were produced in CHO cells stably transfected with the PPI4-tPA-gp120 plasmids and purified to homogeneity as described (Hasel et al. U.S. Pat. Nos. 5,869,624 and 5,886,163; Trkola et al. Nature 384:184, 1996). The antibodies designated PA8, PA10, PA12 and PA14 were prepared by growing the corresponding hybridoma cell line in mouse ascites and isolating the antibody using protein A affinity chromatography as described (Olson et al. J.Virol. 73:4145, 1999). L1.2-CCR5⁺ cells were cultured as described (Olson et al. J.Virol. 73:4145, 1999).

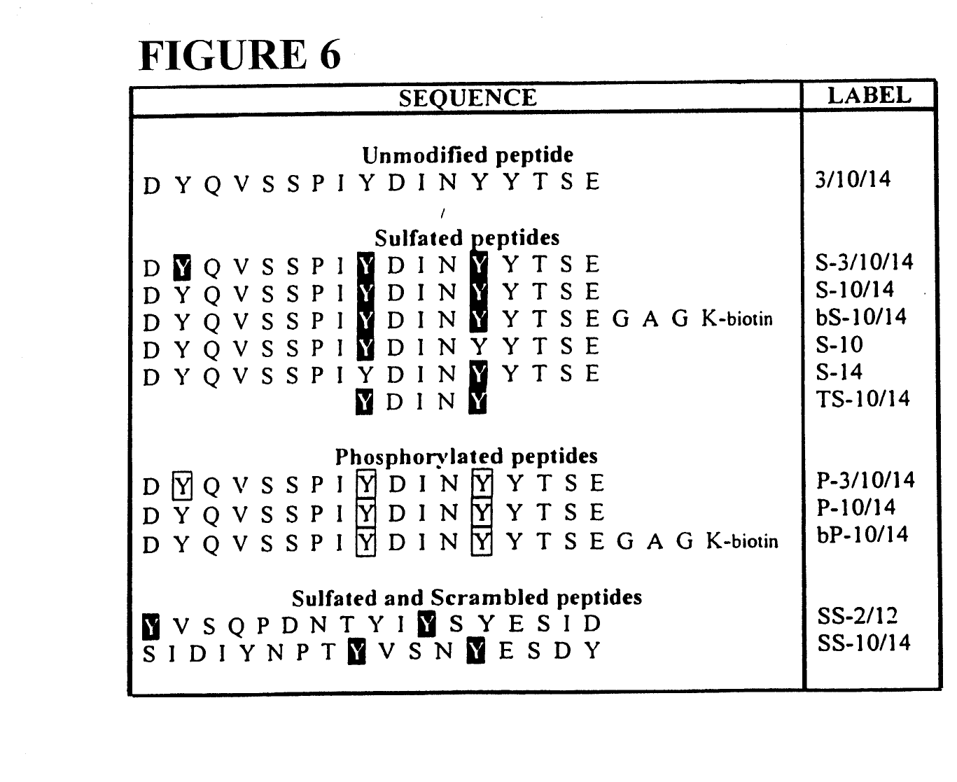

Peptides containing different segments of the CCR5 Nt were custom-synthesized by solid-phase fluorenylmethoxycarbonyl chemistry using phospho- and sulfo-tyrosine precursors as building blocks where indicated (FIG. 6). Biotinylated versions of peptides S-10/14 and P-10/14 incorporated a C-terminal GAG spacer preceding a biotinylated lysine. Following cleavage from the resin, peptides were purified by reverse-phase chromatography on C18 columns (Vydac, Hesperia, Calif.) and analyzed by HPLC and mass spectroscopy. FIG. 6 describes the different peptides that were used in this study.

Binding of gp120 to CCR5

A gp120/CD4 complex formed from monomeric gp120 (100 nM) and biotinylated CD4-IgG2 (50 nM) was added to 1×10⁶ L1.2-CCR5⁺ cells in the presence of different concentrations of peptide (Olson et al. J.Virol. 73:4145, 1999). CD4-IgG2 is tetrameric and therefore binds four molecules of gp120, which increases binding of the complex to CCR5 (Allaway et al. AIDS Res. Hum. Retroviruses 11:533, 1995). The mean fluorescence intensity (m.f.i.) was measured by flow cytometry after addition of phycoerythrin (PE)-labeled streptavidin (Becton Dickinson, San Jose, Calif.). Inhibition of gp120/CCR5 binding was calculated: (m.f.i. with peptide)/(m.f.i. without peptide)×100%.

It was first tested whether tyrosine-sulfated peptides spanning amino acids 2-18 of the CCR5 Nt could inhibit binding of the gp120_{JR-FL}/CD4-IgG2 complex to CCR5⁺ cells.

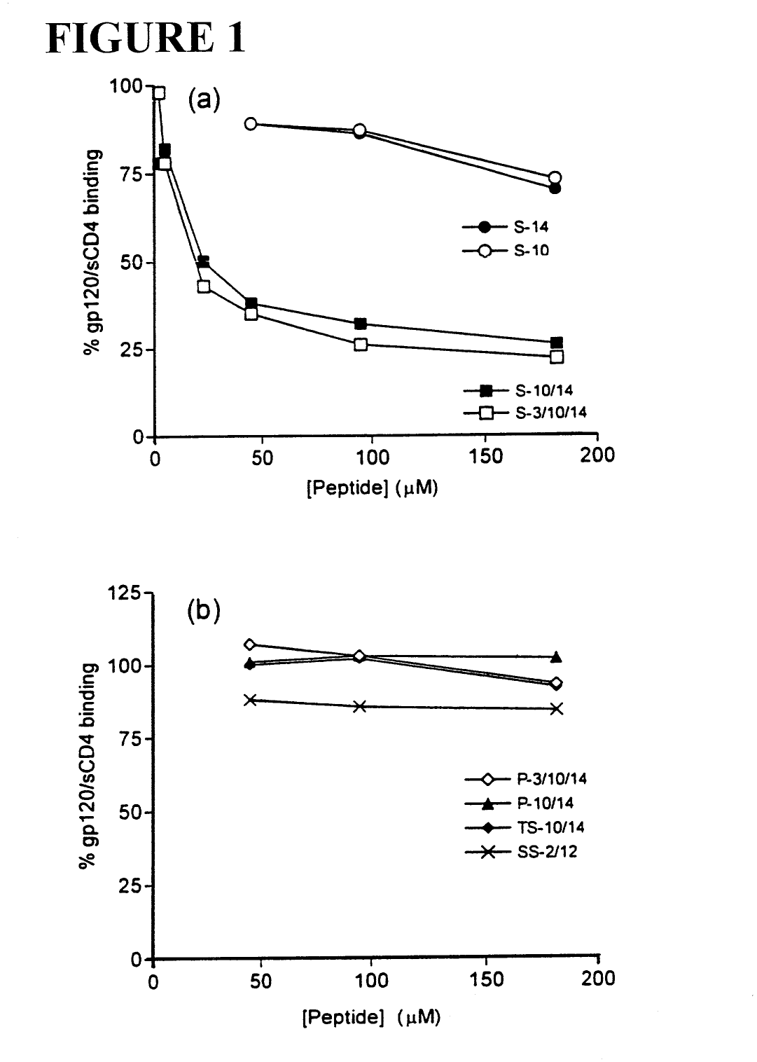

The HIV-1_{JR-FL} isolate exclusively uses CCR5 as a co-receptor (Dragic et al. Nature 381:667, 1996). Only peptides S-3/10/14 and S-10/14 inhibited complex binding to the cells in a dose-dependent manner (FIG. 1a). Peptides S-10 and S-14 had no inhibitory activity, even at the highest concentrations (FIG. 1a). Peptide TS-10/14, spanning amino acids 10-14, did not inhibit gp120_{JR-FL}/CD4-IgG2 binding to CCR5⁺ cells, despite the presence of two sulfo-tyrosine residues (FIG. 1b).

Tyrosine-phosphorylated peptides P-10/14 and P-3/10/14 did not inhibit gp120_{JR-FL}/CD4-IgG2 binding to CCR5⁺ cells (FIG. 1b). As further specificity controls we synthesized peptides containing the first seventeen residues of the CCR5 Nt in random order with sulfo-tyrosines in positions 10 and 14 (SS-10/14) or in positions 2 and 12 (SS-2/12). Neither one of these peptides reduced gp120_{JR-FL}/CD4-IgG2 binding to CCR5⁺ cells, even at the highest concentrations (FIG. 1b).

Surface Plasmon Resonance Measurements (BIAcore)

Streptavidin-coated sensor chips (BIAcore AB, Sweden) were conditioned with five injections of regeneration solution (1M NaCl, 50 mM NaOH) and equilibrated with HBS-EP buffer (10 mM HEPES, 150 mM NaCl, 3M EDTA, 0.005% polysorbate 20) as recommended by the manufacturer. Biotinylated peptides were then immobilized on the chip by injection of peptide (100 nM) in HBS-EP buffer, followed by an injection of regeneration solution and equilibration with HBS-EP buffer. 400 resonance units (RU) of peptide were bound to the sensor chip surface. Solutions of the following proteins (100 nM) were passed over the sensor chip surface: gp120, sCD4, gp120/sCD4, PA8, PA10 and 2D7. Surface plasmon resonance was monitored and displayed in arbitrary resonance units (RU) as a function of time. Following injection of each solution the chip was regenerated and equilibrated as described above.

Biotinylated peptide was attached to the streptavidin-coated gold surface of a sensor chip and solutions containing different gp120/sCD4 complexes were flowed over the immobilized peptide. Adsorption of the complex due to complex/peptide binding was detected by an increase in surface plasmon resonance signal (RU), which reports changes in the effective refraction index very near the gold surface of the sensor chip (Schuck Ann. Rev. Biophys Biomol Struct 26:541, 1997). For proteins of similar size, such as the different gp120/sCD4 complexes, RU plateau values are directly proportional to the amount of protein bound to the peptide.

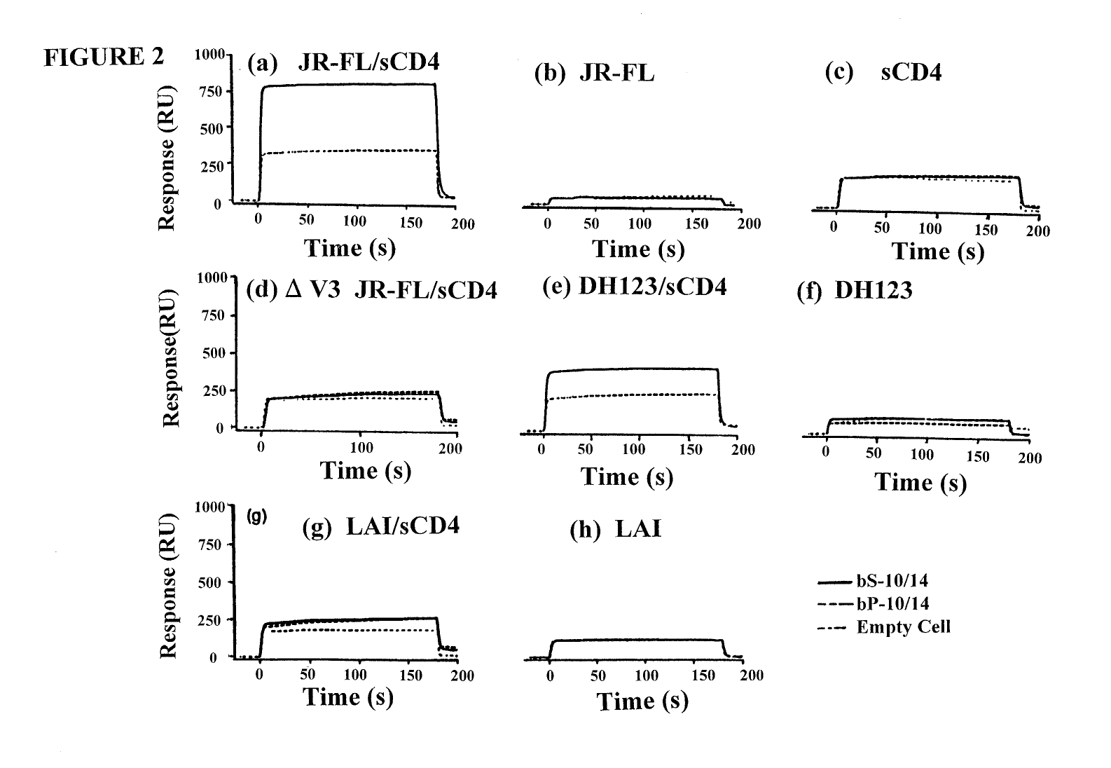

Specific association of the gp120_{JR-FL}/sCD4 complex with the sulfo-tyrosine-containing peptide bS-10/14 was accompanied by a significant increase in RU (FIG. 2a) The signal plateau but not the shape of the sensograms varied with gp120_{JR-FL}/sCD4 concentration indicating that the peptide/complex interaction was dose-dependent (data not shown). The sensorgram obtained with bP-10/14 is similar to the one obtained in the absence of peptide, indicating a complete lack of association of the phosphorylated peptide with the protein complex (FIG. 2a). Neither gp120_{JR-FL} nor sCD4 alone produced a significant increase in RU, indicating that they did not associate with the immobilized peptides. (FIG. 2b,c). The gp120-ΔV3_{JR-FL}/sCD4 complex was also unable to associate with the peptides (FIG. 2d).

To further ascertain the specificity of the peptide/complex association we performed BIAcore analyses using envelope glycoproteins from HIV-1_{DH123}, an R5X4 isolate, and HIV-1_{LAI}, an X4 isolate (5). gp120_{DH123}/sCD4 associated specifically with the sulfated peptide, although the plateau RU values were lower than those observed with gp120_{JR-FL}/sCD4 (FIG. 2e). We did not detect any binding of gp120_{DH123}/sCD4 to the phosphorylated peptide (FIG. 2e), nor did gp120_{DH123} alone associate with the peptides (FIG. 2f). Finally, gp120_{LAI} with or without sCD4 was not able to associate with either one of the peptides (FIG. 2g,h).

These methods could be readily modified to screen for agents that bind CCR5 or that block its interaction with antibodies, gp120 or other ligands. For example, direct binding of the agents could be analyzed as described above, where the agent is substituted for the anti-CCR5 antibody or gp120/sCD4 complex. In another embodiment, the agent could be mixed or pre-incubated with the anti-CCR5 antibody (or gp120/sCD4 complex) prior to passing the mixture over biosensor chips as described above.

Binding of MAbs to CCR5

L1.2-CCR5 cells (1×10⁶) were incubated with anti-CCR5 MAb (50 nM)±peptide (100 μM). MAb binding was detected using a PE-labeled goat anti-mouse antibody (Caltag Laboratories, Burlingam, Calif.). The m.f.i value was measured by flow cytometry as described (Olson et al. J. Virol. 73:4145, 1999). MAb binding was calculated as above.

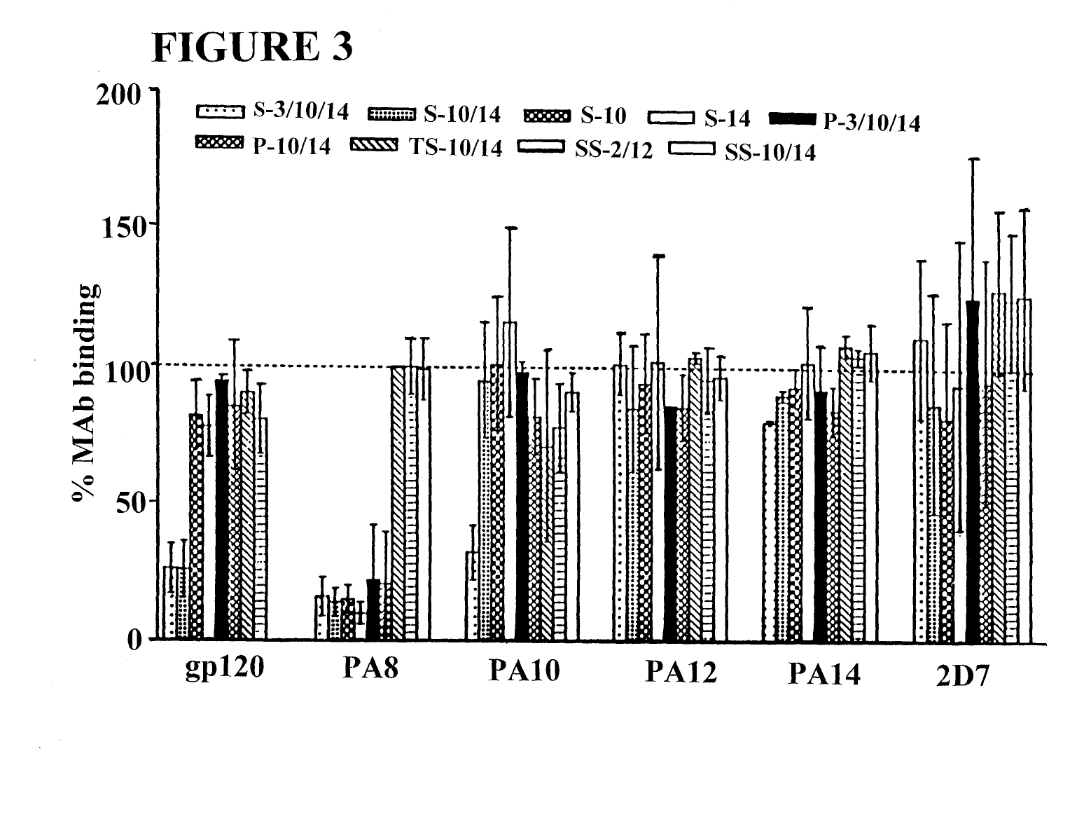

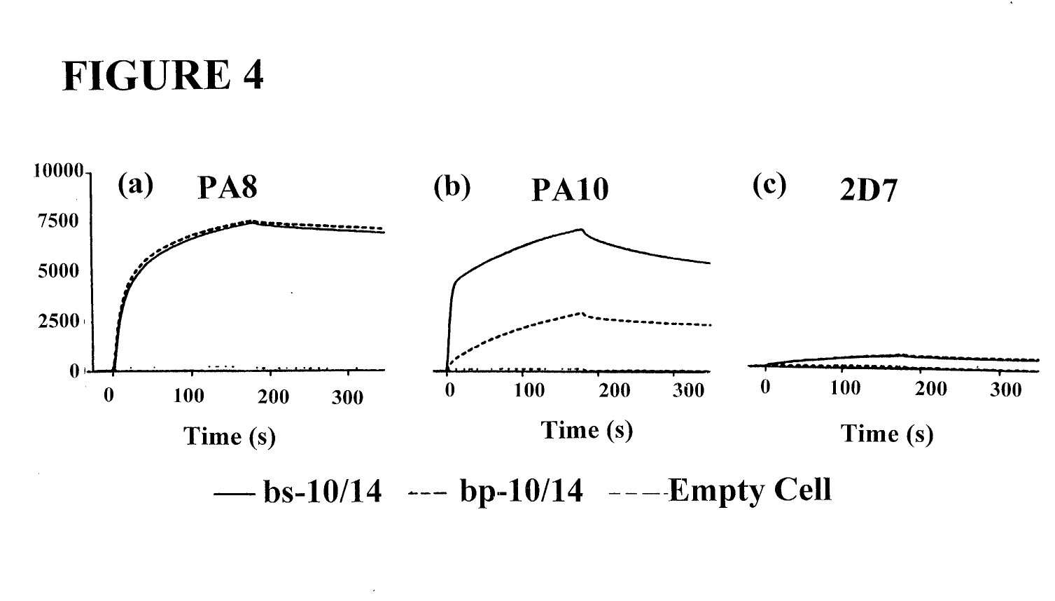

We determined whether the CCR5 Nt peptides could inhibit binding of a panel of anti-CCR5 MAbs to CCR5⁺ cells. PA8 binding was reduced significantly by all wild-type peptides containing amino acids 2-18, regardless of tyrosine modification (FIG. 3). BIAcore analysis confirmed that PA8 similarly and specifically associated with both sulfated and phosphorylated peptides (FIG. 4). Binding of PA12 to CCR5 was not inhibited by any of the peptides (FIG. 3). PA10 binding to CCR5 was inhibited only by S-3/10/14 (FIG. 3). PA10 was also observed to associate with bS-10/14 and to a lesser extent with bP-10/14 in BIAcore analysis (FIG. 4), which may be more sensitive than the gp120/CCR5-binding assay. Binding of 2D7 to CCR5 was not inhibited by any of the peptides (FIG. 3). No significant interaction was observed between any CCR5 Nt peptide and Mab 2D7 (FIGS. 3 and 4), whose epitope resides within the second extracellular loop on CCR5.

Single Cycle HIV-1 Entry Assay

Nlluc⁺env⁻ particles pseudotyped with envelope glycoproteins from MuLV, HTLV-1 and HIV-1 strains JR-FL, HxB₂, DH123, Gun-1 were made as described (Dragic et al. J. Virol. 72:279, 1998). Target cells (Hela-CD4⁺CCR5⁺ or U87-CD4⁺CCR5⁺) were incubated with virus-containing supernatant fractions (100 ng/ml p24)±peptide (100 μM) for 4 h. then washed and resuspended in culture media. After 48 hours the cells were lysed and luciferase activity (relative light units, r.l.u.) was measured using a standard kit (Promega, Madison, Wis.) as described (Dragic et al. J. Virol. 72:279, 1998). Viral entry was calculated: (r.l.u. with peptide)/(r.l.u without peptide)×100%.

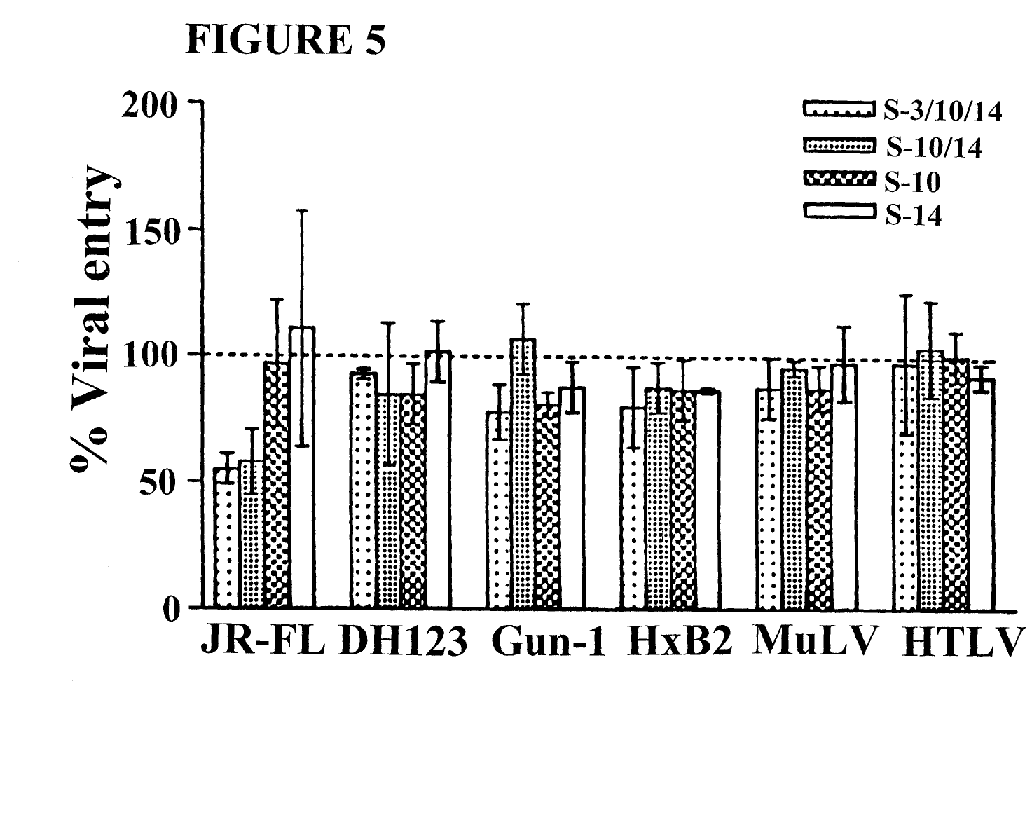

The ability of different CCR5 Nt peptides to inhibit HIV-1 entry into CD4⁺CCR5⁺CXCR4⁺ cells was tested using a luciferase-based single round of entry assay (5). Only peptides S-10/14 and S-3/10/14 inhibited the entry of the R5 isolate HIV-1_{JR-FL} by approximately 50% in HeLa-CD4⁺CCR5⁺ and U87MG-CD4⁺CCR5⁺ (FIG. 5 and data not shown). We were unable to inhibit the entry of the R5X4 isolates HIV-1_{DH123} and HIV-1_{Gun-1}, or of the X4 isolate HIV-1_{HxB2}. The entry of MuLV and HTLV pseudotypes was also unaffected by the peptides (FIG. 5).

Screening Assays

1) HIV-1 gp120/CD4-IgG2

Streptavidin-coated 96-well microtiter plates (NEN Life Science Products, Boston, Mass.) are blocked with 200 μl/well of 5% bovine serum albumin (Sigma, St. Louis, Mo.) in PBS buffer and washed with assay buffer (0.5% Tween 20, 1% fetal bovine serum, and 2% BSA in PBS buffer). The plates are then incubated 1 hour at ambient temperature with 100 μl/well of biotinylated CCR5 N-terminal sulfopeptide at a concentration of 500 μM in assay buffer. Following a wash step, the plates are incubated for 1 hour at ambient temperature with an HIV-1_{JR-FL} gp120/CD4-IgG2 complex in the presence or absence of inhibitory agent. The plates are again washed and incubated for 30 minutes with a horseradish peroxidase-labeled goat antibody to human IgG (Kirkegaard & Perry Laboratories, Gaithersburg, Md.) followed by addition of the TMB (3,3′,5,5′-tetramethylbenzidine) chromogenic substrate (Pierce). The reaction is stopped by addition of 100 μl/well of 2N H₂SO₄ prior to calorimetric detection at a wavelength of 450 nm. Wells without biotinylated peptide serve as negative controls. The percent inhibition of binding is calculated as [1−(OD_{with inhibitor}−OD_{control well})/(OD_{without inhibitor}−OD_{control well})]×100, where OD represents the average optical density observed for the indicated wells.

2) Anti-CCR5 Antibodies

Streptavidin-coated microtiter plates are blocked and incubated with CCR5 N-terminal peptide as described above. Following a wash step, the plates are incubated for one hour at ambient temperature with the anti-CCR5 antibody PA10 in the presence or absence of inhibitory agent. The plates are again washed and incubated for 30 minutes with a horseradish peroxidase-labeled goat antibody to mouse IgG (Kirkegaard & Perry Laboratories, Gaithersburg, Md.) followed by addition of TMB substrate for calorimetric detection as described above. The percent inhibition mediated by the inhibitory agent is calculated as described above.

Discussion

Tyrosine-modified peptides spanning the region of the CCR5 Nt that contains residues important for viral entry were synthesized. (Dragic et al. J. Virol. 72:279, 1998; Rabut et al. J. Virol. 72:3464, 1998; Farzan et al. J. Virol. 72:1160, 1998; Dorantz et al. J. Virol. 71:6305, 1997). Interactions between the Nt peptides and gp120/CD4 complexes were characterized. Peptides containing sulfo-tyrosines in positions 10 and 14 efficiently inhibited binding of gp120_{JR-FL}/CD4 to CCR5. Substitution of the sulfate groups for phosphates, which are also negatively charged at physiological pH, rendered the Nt peptides inactive. Inhibition of gp120/CCR5 binding was dependent, therefore, on the presence of sulfate moieties and was not simply due to non-specific electrostatic interactions between the peptide and the gp120/CD4 complex or the peptide and the cell surface. Inhibition of gp120/CCR5 binding was also dependent on the primary structure surrounding the sulfo-tyrosines since peptides with random sequences of CCR5 amino acids 2-18 had no inhibitory activity. Additional Nt amino acids in the region 2-18 were important for activity since a shortened peptide containing just amino acids 10-14 was unable to inhibit gp120/CD4 binding, despite the presence of two sulfo-tyrosines. It would be straightforward to define the minimum number of amino acids needed for activity by systematically synthesizing sulfopeptides intermediate in length between peptide 2-18 and peptide 10-14. Similarly, sulfopeptides that incorporate a greater portion of the CCR5 Nt could be easily synthesized and tested for activity using the methods described herein.

Qualitative BIAcore analyses allowed the demonstration of a highly specific, CD4-dependent interaction between a tyrosine-sulfated Nt peptide and gp120_{JR-FL}. No binding of the protein complex to a tyrosine-phosphorylated peptide was observed. Only gp120s derived from isolates that use CCR5 as a co-receptor associated with the sulfated peptide. gp120_{DH123}/CD4 binding was weaker than gp120_{JR-FL}/CD4 binding, suggesting that envelope glycoproteins from R5X4 isolates have a lower apparent affinity for CCR5 than envelope glycoproteins from R5 isolates. gp120_{LAI}, derived from an isolate that only uses CXCR4, did not bind to the sulfated peptide. A V3 loop-deleted gp120_{JR-FL} did not associate with the sulfated peptide, just as this protein was unable to bind to full length CCR5 on the cell surface (Trkola et al. Nature 384:184, 1996).

The binding of the Nt peptides to several anti-CCR5 MAbs, all of which recognize conformational epitopes in CCR5 and inhibit gp120/CCR5 binding were also studied. PA12 and 2D7 did not bind to any of the peptides. Binding of PA8 to the peptides was independent of tyrosine-modification whereas PA10 associated more with the sulfo-tyrosine-containing peptide than with the phospho-tyrosine-containing peptide. It seems, therefore, that sulfo-tyrosines and phospho-tyrosines are relatively interchangeable for the purpose of MAb binding but that gp120/CD4 binding has an absolute requirement for sulfo-tyrosines. Relatively subtle differences in size and geometry of sulfate and phosphate groups might be relevant for binding of the CCR5 Nt with gp120, which must not only accept the negative charge, but also coordinate, probably by hydrogen bonds, the tyrosine sulfate oxygens. The kinetics of MAb binding to the CCR5 Nt peptides exhibited large apparent on rates and slow apparent off rates, which also differed from our observations of gp120/CD4 binding kinetics.

None of the Nt peptides inhibited MuLV, HTLV and HIV-1_{HxB2} envelope-mediated viral entry, which is not mediated by CCR5. In contrast, peptides S-10/14 and S-3/10/14 specifically inhibited the entry of the HIV-1_{JR-FL} R5 strain in two different cell lines. The inhibition of HIV-1 entry by tyrosine-sulfated peptides was partial (˜50%) but nonetheless striking given the difficulty of blocking this process with short, linear peptides (Jameson et al. Science 240:1335, 1988; Chan and Kim Cell 93:681:1998; Doranz et al. J. Exp. Med. 186:1395, 1997; Heveker et al. Current Biology 8:369, 1998; Eckert et al. Cell 99:1, 1999).

### REFERENCES

1. E. A. Berger et al. (1999) Annu Rev Immunol 17: 657-700.

2. A. Trkola et al. (1996) Nature 384: 184-187.

3. L. Wu et al. (1996) Nature 384: 179-183.

4. Y. J. Zhang et al. (1999) J. Virol 73: 3443-3448.

5. T. Dragic et al. (1998) J. Virol. 72: 279-285.

6. G. E. Rabut et al. (1998) J. Virol. 72:3464-3468.

7. M. Farzan et al. (1998) J. Virol. 72:1160-1164.

8. B. J. Doranz et al. (1997) J. virol. 71: 6305-6314.

9. M. Farzan et al. (1999) Cell 96:667-676.

10. P. A. Baeuerle et al. (1987) J. Cell Biol 105:2655-2664.

11. J. W. Kehoe et al. (2000) Chemistry & Biology 7:R57-R61.

12. M. Baba et al. (1988) Proc Natl Acad Sci USA 85: 6132-6136.

13. D. Schols et al. (1990) Virology 175: 556-561.

14. G. Roderiquez (1995) J. Virol 69: 2233-2239.

15. S. S. Hwang et al. (1991) Science 253: 71-74.

16. F. Safaiyan et al. (1999) J. Biol Chem 274: 36267-36273.

17. P. A. Baeuerle et al. (1986) Biochem Biophys Res Commun 141: 870-877.

18. W. C. Olson et al. (1999) J Virol 73:4145-4155.

19. G. P. Allaway et al. (1995) AIDs Res Hum Retroviruses 11: 533-539.

20. T. Dragic et al. (1996) Nature 381:667-673.

21. P. Schuck et al. (1997) Annu Rev Biophys Biomol Struct 26:541-566.

22. S. Lederman et al. (1989) J. Immunol 143:1149-1154.

23. S. Ohlson et al. (2000) Trends in Biotechnology 18: 49-52.

24. B. A. Jameson et al. (1988) Science 240: 1335-1339.

25. D. C. Chan et al. (1998) Cell 93: 681-684.

26. B. J. Doranz et al. (1997) J. Exp Med 186: 1395-1400.

27. N. Heveker et al. (1998) Current Biology 8:369-376.

28. D. M. Eckert et al. (1999) Cell 99: 1-20.

29. H. Sakaida et al. (1998) J. Virol 72: 9763-9770.

30. S. E. Kuhman et al. (1997) J. Virol 71: 8642-8656.

31. T. M. Ross et al. (1998) J. Virol 72: 1918-1924.

32. R. E. Atchison et al. (1996) Science 274: 1924-1926.

33. P. D. Bieniasz et al. (1997) EMBO 16: 2599-2609.

34. A. L. Edinger et al. (1999) J. Virol 73:4062-4073.

35. J. Rucker et al. (1996) Cell 87: 437-446.

36. L. Picard et al. (1997) J. Virol 71: 5003-5011.

37. T. Dragic et al. (2000) Proc Natl Acad Sci 10: 1073.

38. P. D. Kwong et al. (1998) Nature 393: 648-659.

39. C. D. Rizzuto et al. (1998) Science 280: 1949-1953.

40. P. D. Kwong et al. (2000) J. Virol 74: 1961-1972.

41. M. Moulard et al. (2000) J. Virol 74: 1948-1960.

42. A. Trkola et al. (1996) Nature 384: 184-186.

43. B. Labrosse et al. (1998) J. Virol 72: 6381-6388.

44. Spetzler et al. (1995) J. Pept. Prot Res 45: 78.

45. Yai et al. Et al. (1995) J. Virol 69: 320.

46. Okuda et al. (1993) J. Mol. Recognit. 6:101.

47. Sabatier et al. (1993) Biochemistry 32: 2763.

What is claimed:

1. A compound comprising the structure:
θαYDINYYTSβλ
wherein each Y represents a tyrosine; each D represents an aspartic acid, each I represents an isoleucine; and each N represents an asparagine;
wherein α represents from 0 to 9 amino acids, with the proviso that if there are more than 2 amino acids, they are joined by peptide bonds in consecutive order and have a sequence identical to the sequence set forth in SEQ ID NO: 1 beginning with the I at position 9 and extending therefrom in the amino terminal direction;
wherein β represents from 0 to 14 amino acids, with the proviso that if there are more than 2 amino acids, they are joined by peptide bonds in consecutive order and have a sequence identical to the sequence set forth in SEQ ID NO: 1 beginning with the E at position 18 and extending therefrom in the carboxy terminal direction;
wherein θ represents an amino group or an acetylated amino group; wherein λ represents a carboxyl group or an amidated carboxyl group;
wherein all of α,Y,D,I,N,Y,Y,T,S and β are joined together by peptide bonds;
further provided that at least two tyrosines in the compound are sulfated.

2. The compound of claim 1, wherein β represents less than 17 amino acids.

3. The compound of claim 1, wherein β represents less than 16 amino acids.

4. The compound of claim 1, wherein β represents less than 15 amino acids.

5. The compound of claim 1, wherein β represents less 10 than 14 amino acids.

6. The compound of claim 1, wherein β represents less than 13 amino acids.

7. The compound of claim 1, wherein β represents less than 12 amino acids.

8. The compound of claim 1, wherein β represents less than 11 amino acids.

9. The compound of claim 1, wherein β represents less than 10 amino acids.

10. The compound of claim 1, wherein β represents less than 9 amino acids.

11. The compound of claim 1, wherein β represents less than 8 amino acids.

12. The compound of claim 1, wherein β represents less than 7 amino acids.

13. The compound of claim 1, wherein β represents less than 6 amino acids.

14. The compound of claim 1, wherein β represents less than 5 amino acids.

15. The compound of claim 1, wherein β represents less than 4 amino acids.

16. The compound of claim 1, wherein β represents less than 3 amino acids.

17. The compound of claim 1, wherein β represents less than 2 amino acids.

18. The compound of claim 1, wherein β represents less than 1 amino acid.

19. The compound of claim 1, wherein α represents less than 9 amino acids.

20. The compound of claim 1, wherein α represents less than 8 amino acids.

21. The compound of claim 1, wherein α represents less than 7 amino acids.

22. The compound of claim 1, wherein α represents less than 6 amino acids.

23. The compound of claim 1, wherein α represents less than 5 amino acids.

24. The compound of claim 1, wherein α represents less than 4 amino acids.

25. The compound of claim 1, wherein α represents less than 3 amino acids.

26. The compound of claim 1, wherein α represents less than 2 amino acids.

27. The compound of claim 1, wherein α represents less than 1 amino acid.

28. A composition comprising the compound of claim 1 and a detectable marker attached thereto.

29. The composition of claim 28, wherein the detectable marker is biotin.

30. The composition of claim 28, wherein the detectable marker is attached at the C-terminus of the compound.

31. A composition which comprises a carrier and an amount of the compound of claim 1 effective to inhibit binding of HIV-1 to a CCR5 receptor on the surface of a CD4+ cell.

32. A method of inhibiting human immunodeficiency virus infection of a CD4+ cell which also carries a CCR5 receptor on its surface which comprises contacting the CD4+ cell with an amount of the compound of claim 1 effective to inhibit binding of human immunodeficiency virus to the CCR5 receptor so as to thereby inhibit human immunodeficiency virus infection of the CD4+ cell.

33. The method of claim 32, wherein the CD4+ cell is present in a subject and the contacting is effected by administering the compound to the subject.

34. A method of preventing CD4+ cells of a subject from becoming infected with human immunodeficiency virus which comprises administering to the subject an amount of the compound of claim 1 effective to inhibit binding of human immunodeficiency virus to CCR5 receptors on the surface of the CD4+ cells so as to thereby prevent the subject's CD4+ cells from becoming infected with human immunodeficiency virus.

35. A method of treating a subject whose CD4+ cells are infected with human immunodeficiency virus which comprises administering to the subject an amount of the compound of claim 1 effective to inhibit binding of human immunodeficiency virus to CCR5 receptors on the surface of the subject's CD4+ cells so as to thereby treat the subject.

36. The method of any one of claims 33-35, wherein the compound is administered by aerosol, intravenous, oral or topical route.

37. The method of claim 33 or 35, wherein the subject is infected with HIV-1 prior to administering the compound to the subject.

38. The method of claim 33 or 34, wherein the subject is not infected with HIV-1 prior to administering the compound to the subject.

39. The method of claim 38, wherein the subject is not infected with, but has been exposed to, human immunodeficiency virus.

40. The method of any one of claims 33-35, wherein the effective amount of the compound comprises from about 1.0 ng/kg to about 100 mg/kg body weight of the subject.

41. The method of claim 40, wherein the effective amount of the compound comprises from about 100 ng/kg to about 50 mg/kg body weight of the subject.

42. The method of claim 41, wherein the effective amount of the compound comprises from about 1 μg/kg to about 10 mg/kg body weight of the subject.

43. The method of claim 42, wherein the effective amount of the compound comprises from about 100 μg/kg to about 1 mg/kg body weight of the subject.

44. The method of any one of claims 33-35, wherein the subject is a human being.

45. A method of identifying an agent which inhibits binding of a CCR5 ligand to a CCR5 receptor which comprises:
(a) immobilizing the compound of claim 1 on a solid support;
(b) contacting the immobilized compound from step (a) with sufficient detectable CCR5 ligand to saturate all binding sites for the CCR5 ligand on the immobilized compound under conditions permitting binding of the CCR5 ligand to the immobilized compound so as to form a complex;
(c) removing any unbound CCR5 ligand;
(d) contacting the complex from step (b) with the agent; and
(e) detecting whether any CCR5 ligand is displaced from the complex, wherein displacement of detectable CCR5 ligand from the complex indicates that the agent binds to the compound so as to thereby identify the agent as one which inhibits binding of the CCR5 ligand to the CCR5 receptor.

46. A method of identifying an agent which inhibits binding of a CCR5 ligand to a CCR5 receptor which comprises:
(a) contacting the compound of claim 1 with sufficient detectable CCR5 ligand to saturate all binding sites for the CCR5 ligand on the compound under conditions permitting binding of the CCR5 ligand to the compound so as to form a complex;
(b) removing any unbound CCR5 ligand;
(c) measuring the amount of CCR5 ligand which is bound to the compound in the complex;
(d) contacting the complex from step (a) with the agent so as to displace CCR5 ligand from the complex;
(e) measuring the amount of CCR5 ligand which is bound to the compound in the presence of the agent; and
(f) comparing the amount of CCR5 ligand bound to the compound in step (e) with the amount measured in step (c), wherein a reduced amount measured in step (e) indicates that the agent binds to the compound so as to thereby identify the agent as one which inhibits binding of the CCR5 ligand to the CCR5 receptor.

47. A method of identifying an agent which inhibits binding of a CCR5 ligand to a CCR5 receptor which comprises:
(a) immobilizing the compound of claim 1 on on a solid support;
(b) contacting the immobilized compound from step (a) with the agent and detectable CCR5 ligand under conditions permitting binding of the CCR5 ligand to the immobilized compound so as to form a complex;
(c) removing any unbound CCR5 ligand;
(d) measuring the amount of detectable CCR5 ligand which is bound to the immobilized compound in the complex;
(e) measuring the amount of detectable CCR5 ligand which binds to the immobilized compound in the absence of the agent;
(f) comparing the amount of CCR5 ligand which is bound to the immobilized compound in step (e) with the amount measured in step (d), wherein a reduced amount measured in step (d) indicates that the agent binds to the compound or CCR5 ligand so as to thereby identify the agent as one which inhibits binding of the CCR5 ligand to the CCR5 receptor.

48. The method of claim 47, wherein the amount of the detectable ligand in step (a) and step (e) is sufficient to saturate all binding sites for the CCR5 ligand on the compound.

49. A method of identifying an agent which inhibits binding of a CCR5 ligand to a CCR5 receptor which comprises:
(a) contacting the compound of claim 1 with the agent and detectable CCR5 ligand under conditions permitting binding of the CCR5 ligand to the compound so as to form a complex;
(b) removing any unbound CCR5 ligand;
(c) measuring the amount of detectable CCR5 ligand which is bound to the compound in the complex;
(d) measuring the amount of detectable CCR5 ligand which binds to the compound in the absence of the agent;
(e) comparing the amount of CCR5 ligand which is bound to the compound in step (c) with the amount measured in step (d), wherein a reduced amount measured in step (c) indicates that the agent binds to the compound or CCR5 ligand so as to thereby identify the agent as one which inhibits binding of the CCR5 ligand to the CCR5 receptor.

50. The method of claim 49, wherein the amount of the detectable ligand in step (a) and step (d) is sufficient to saturate all binding sites for the CCR5 ligand on the compound.

51. The method of any one of claims 45-50, wherein the detectable CCR5 ligand is labeled with a detectable marker.

52. A method of identifying an agent which inhibits binding of a CCR5 ligand to a CCR5 receptor which comprises:
a) immobilizing the compound of claim 1 on a solid support;
b) contacting the immobilized compound from step a) with the agent dissolved or suspended in a known vehicle and measuring the binding signal generated by such contact;
c) contacting the immobilized compound from step a) with the known vehicle in the absence of the compound and measuring the binding signal generated by such contact;
d) comparing the binding signal measured in step b) with the binding signal measured in step c), wherein an increased amount measured in step b) indicates that the agent binds to the compound so as to thereby identify the agent as one which binds to the CCR5 receptor.

53. The method of claim 52, wherein the solid support is a surface plasmon resonance sensor chip.

54. The method of claim 52 or 53, wherein the binding signal is measured by surface plasmon resonance.

55. A method of obtaining a composition which comprises:
(a) identifying a compound which inhibits binding of a CCR5 ligand to a CCR5 receptor according to the method of any one of claims 45-50 and 52; and
(b) admixing the compound so identified or a homolog or derivative thereof with a carrier.

56. The method of any one of claims 45-50 and 52, wherein the CCR5 ligand is a complex comprising an HIV-1 envelope glycoprotein and a CD4-based protein.

57. The method of claim 56, wherein the HIV-1 envelope glycoprotein is gp120, gp140 or gp160.

58. The method of claim 56, wherein the CD4-based protein is soluble CD4 or CD4-IgG2.

59. The method of any one of claims 45-50 and 52, wherein the CCR5 ligand is a chemokine.

60. The method of claim 59, wherein the chemokine is RANTES, MIP-1α or MIP-1β.

61. The method of any one of claims 45-50 and 52, wherein the CCR5 ligand is an antibody.

62. The method of claim 61, wherein the antibody is selected from the group consisting of PA8 (ATCC Accession No. HB-12605), PA10 (ATCC Accession No.12607), PA11 (ATCC Accession No. HB-12608), PA12 (ATCC Accession No. HB-12609).

63. The method of claim 45 or 47, wherein the solid support is a microtiter plate well, a bead or surface plasmon resonance sensor chip.

64. A compound having the structure:
Δ-(αYDINYYTSβλ)_{π}
wherein each T represents a threonine, each S represents a serine, each Y represents a tyrosine; each D represents an aspartic acid, each I represents an isoleucine; and each N represents an asparagine;
wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction;
wherein β represents from 0 to 14 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction;
wherein λ represents a carboxyl group or an amidated carboxyl group;
wherein all of α,Y,D,I,N,Y,Y,T,S and β are joined together by peptide bonds,
further provided that at least two tyrosines in the compound are sulfated,
wherein π is an integer from 1 to 8, Δ is a polymer, and the solid line represents up to 8 linkers which attach the structure in parentheses to Δ.

65. A compound having the structure:
(θαYDINYYTSβ)_{π}-Δ
wherein each T represents a threonine, each S represents a serine, each Y represents a tyrosine; each D represents an aspartic acid, each I represents an isoleucine; and each N represents an asparagine;
wherein α represents from 0 to 9 amino acids, with the proviso that if there are more than 2 amino acids, they are joined together by peptide bonds in consecutive order and have a sequence identical to the sequence set forth in SEQ ID NO: 1 beginning with the I at position 9 and extending therefrom in the amino terminal direction;
wherein β represents from 0 to 14 amino acids, with the proviso that if there are more than 2 amino acids, they are joined together by peptide bonds in consecutive order and have a sequence identical to the sequence set forth in SEQ ID NO: 1 beginning with the E at position 18 and extending therefrom in the carboxy terminal direction;
wherein θ represents an amino group or an acetylated amino group;
wherein all of α,Y,D,I,N,Y,Y,T,S and β are joined together by peptide bonds,
further provided that at least two tyrosines in the compound are sulfated,
wherein π is an integer from 1 to 8, Δ is a polymer, and the solid line represents up to 8 linkers which attach the structure in parentheses to Δ.

66. The compound of claim 64 or 65, wherein the polymer is selected from the group consisting of a linear lysine polymer, a branched lysine polymer, a linear arginine polymer, a branched arginine polymer, polyethylene glycol, a linear acetylated lysine polymer, a branched acetylated lysine polymer, a linear chloroacetylated lysine polymer and a branched chloroacetylated lysine polymer.
